(19) United States
(12) Patent Application Publication
(10) Pub. No.: US 2005/0043582 A1
(43) Pub. Date: Feb. 24, 2005

(54) Surgical instrument having an increased range of motion

(76) Inventor: Michael J. Stokes, Cincinnati, OH (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 10/869,352

(22) Filed: Jun. 16, 2004

Related U.S. Application Data
(60) Provisional application No. 60/478,989, filed on Jun. 17, 2003.

(51) Int. Cl. A61B 1/00

(52) U.S. Cl. 600/101

(57) ABSTRACT

Described is a surgical instrument, such as an endoscopic instrument having at its distal end a working element such as a dissector, scissor or grasper, a shaft that defines a longitudinal axis of the instrument at a handle portion at the proximal end. The shaft rotates about the handle portion of the instrument. The endoscopic instrument also provides for angulation or articulation of the end effector with respect to the longitudinal axis of the shaft. The angulation is accomplished by providing two four bar linkage assemblies connecting the handle portion and the distal end portion of the instrument. A torsion bar provides support to the four bar linkage assembly and provides a lumen for accepting a control cable for actuating the working element.

### CROSS REFERENCE TO RELATED APPLICATION

The present application claims the priority benefit of U.S. provisional patent application Ser. No. 60/478,989, filed on Jun. 17, 2003, which is incorporated herein by reference in its entirety.

### FIELD OF THE INVENTION

The present invention relates in general to surgical instruments, and, more particularly, to a surgical instrument having an increased range of angular motion.

### BACKGROUND OF THE INVENTION

Within endoscopic surgery, there is a recognized need for simple devices such as graspers, dissectors, scissors, and other basic surgical instruments. These instruments are necessary in order to perform simple functions during the endoscopic procedure. Specifically, devices, such as graspers, are necessary in order to properly move the work site away from the other vital organs so that the tissue to be worked on may be isolated and surgery may be performed. Scissors may be needed in order to make an appropriate cut in tissue, muscle or other vasculature. Dissectors can be necessary to separate one portion of tissue from another. These instruments also enable the other, larger instruments such as staplers and ligating clip appliers to have enough volumetric room in order to perform operations such as appendectomies, cholecystectomies, herniorrhaphies, etc.

Traditionally, instruments such as graspers, dissectors, scissors and other endoscopic instruments have been mounted on generally straight shafts. These shafts may or may not have been able to rotate about the longitudinal axis of the shaft. Nonetheless, there has been perceived a need for the end effector of the shaft to be able to angulate with respect to the longitudinal axis of the shaft. This may enable the surgeon to attack the tissue that is to be operated from an oblique angle. In fact, it may be desirable to have the shaft angulate up to 90 degrees. with respect to the longitudinal axis of the shaft. In many ways, this function can be analogized to the capability of the human hand to rotate around the “axis” of the arm, and also “angulate” about the wrist. Of course, while the hand is able to function with pure rotation, the degrees of freedom given by wrist action are much greater and in many ways enhance the ability of the hand to perform simple daily functions. Thus, there is perceived a need for an articulating, angulating endoscopic instrument so that the functions of such mechanisms can be made much more versatile. Particular need is evident in the area of endoscopic suturing and knot tying.

Endoscopic suturing and knot tying prove to be technically challenging for the surgeon. Lack of dexterity, accessibility and stereoscopic visibility all add to the complications for these tasks. Various devices have been developed over the years in order to aid in either interrupted or continuous suturing, as well as knot tying. The surgeon first needs to introduce the needle within the cavity, properly orient the needle in the jaws of the needle driver—usually with a second device, align the end effector of the needle to the tissue—sometimes moving the tissues to the needle, and drive the needle thru the tissue—being careful not to tear the tissue if the needle is not oriented perpendicular to the axis of the driver.

Intracorporeal knot tying is difficult because needle drivers, or graspers, lack maneuverability within the body. To tie a knot, a surgeon grasps the needle within the jaws at the end of relatively long, straight graspers inserted through a trocar. The surgeon needs to pass the needle to another set of relatively long, straight graspers. The axes of the graspers tend to be substantially parallel to each other, making passing a needle from one set of jaws to another through tissue a difficult task. Aligning the axes of the graspers and arranging the jaws of the graspers so that the distal ends can face each other would greatly simplify knot tying. Graspers having jaws that can articulate to create an angle with respect to the axis of the grasper and can rotate on the jaws' own axis would increase the surgeon's maneuverability and greatly simplify knot tying. A surgeon using graspers with these capabilities can angle the jaws to face each other and more easily pass a needle from one set of jaws to another.

Consequently, a significant need exists for a device that provides for more angular movement of the jaws.

### BRIEF SUMMARY OF THE INVENTION

The surgical instrument, such as an endoscopic instrument allows complete control of an end effector, such as a distal jaw or needle holder. The endoscopic instrument uses two perpendicular four bar linkages to articulate the end effector up to + and −45 degrees in two orthogonal planes with respect to the axis of the grasper. The instrument is also capable of rotating the end effector on it's own axis even when the jaws are articulated at an angle with respect to the main axis. The rotation and articulation will allow the surgeon to easily and more accurately position the end effector of the needle prior to penetrating the tissue. Also, since the instrument allows for more control of the end effector, intracorporial knot tying will be easier for the surgeon. The surgeon can use standard size needle and suture combinations. The instrument gives the surgeon an intuitive control for articulation as well as rotation and does not have additional levers or knobs to activate.

Described herein is a surgical instrument having an end effector, such as a dissector, scissor or grasper, having an increased angular motion; a handle having a proximal end and a distal end; a first connector element operatively attached to the end effector; a second connector element operatively attached to the distal end of the handle; and a first and second pair of connector rods connecting the first connector element to the second connector element and forming a first and second four bar linkage assembly. Preferably, the first and second pair of connector rods are pivotally connected to the first and second connector element. The invention also provides for a torsion bar disposed between the first and second connector elements and comprising two pair of channels for operatively accepting the first and second pair of connector rods.

The present invention is useful in open or endoscopic surgeries as well as robotic-assisted surgeries.

Further features and advantages of the present invention will become readily apparent from the following detailed description, the accompanying drawings, and the appended claims.

### BRIEF DESCRIPTION OF THE FIGURES

FIG. 1 is an elevation view of a surgical grasper embodying the principles of the present invention;

FIG. 2 is an enlarged view of the distal end of a surgical grasper illustrating the two sets of four bar linkage configuration;

FIG. 3 is a cross-sectional view of the torsion bar for use in the present invention;

FIG. 4 is a side view of one embodiment of the grasper portion of the present invention;

FIG. 5 is a view of one embodiment of the proximal end or handle portion of the present invention;

FIG. 6 is a perspective view of an alternate embodiment of the proximal end or handle portion of the present invention;

FIG. 7 is a perspective view of the underside of the handle portion of FIG. 6;

FIG. 8 is a cut away view of the handle portion of FIG. 6 illustrating the rack and pinion configuration; and

FIG. 9 is a schematic view of the rack and pinion configuration.

### DETAILED DESCRIPTION OF THE INVENTION

Before explaining the present invention in detail, it should be noted that the invention is not limited in its application or use to the details of construction and arrangement of parts illustrated in the accompanying drawings and description. The illustrative embodiments of the invention may be implemented or incorporated in other embodiments, variations and modifications, and may be practiced or carried out in various ways. Furthermore, unless otherwise indicated, the terms and expressions employed herein have been chosen for the purpose of describing the illustrative embodiments of the present invention for the convenience of the reader and are not for the purpose of limiting the invention.

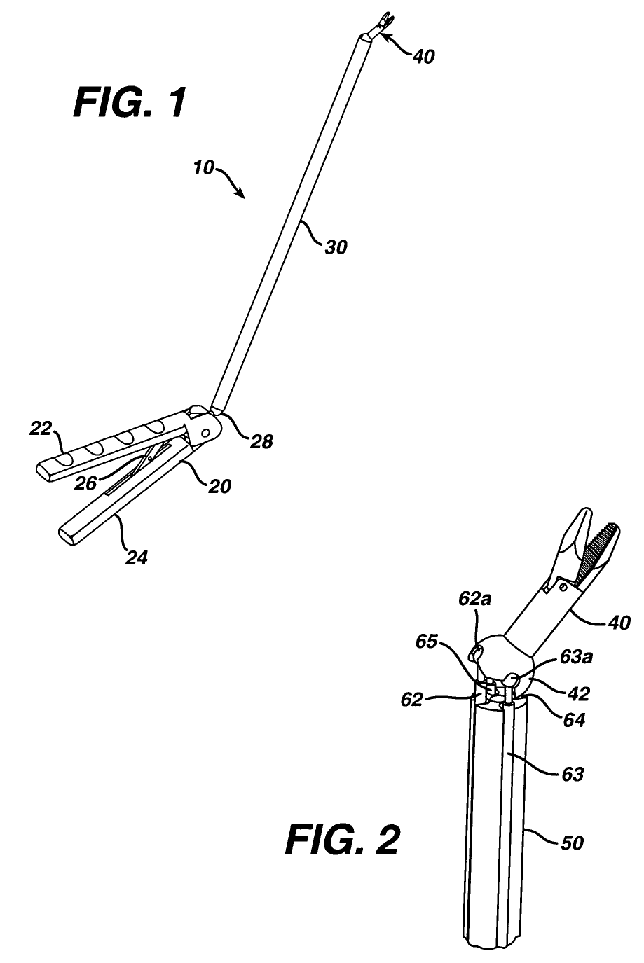

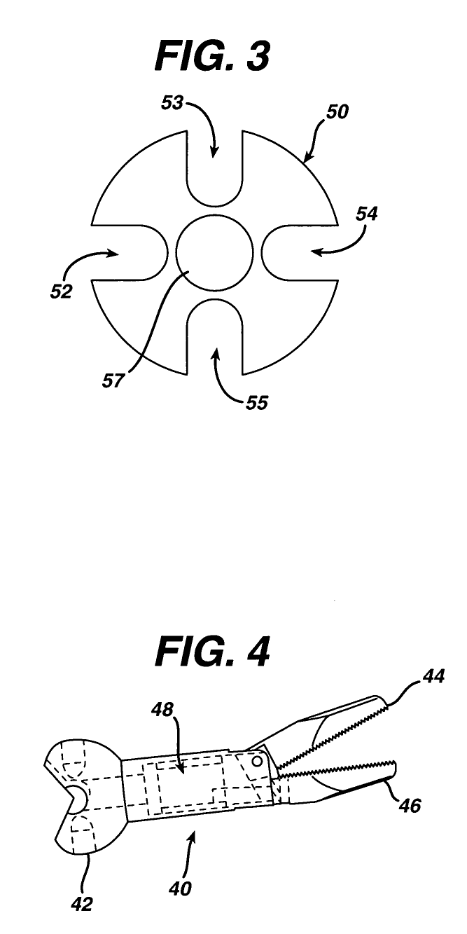

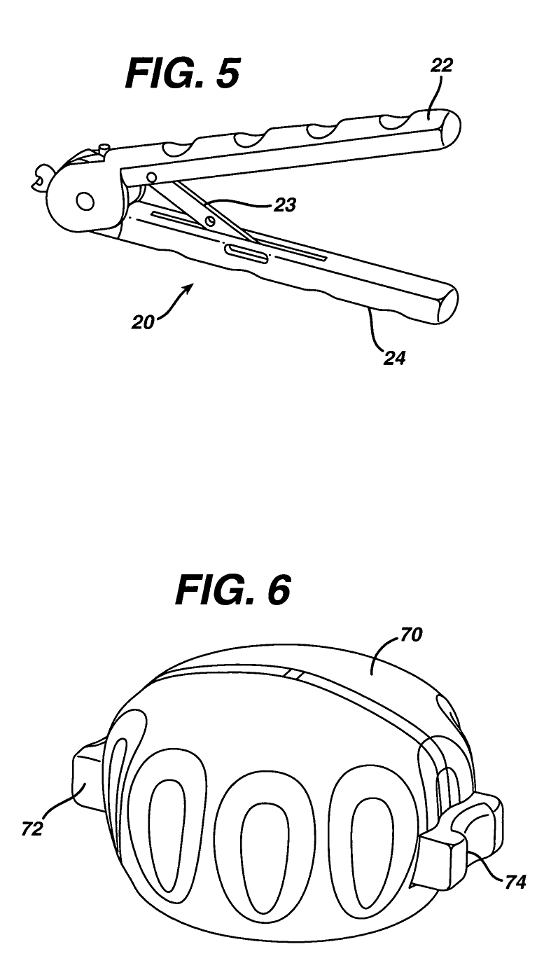

Referring now to FIG. 1, surgical grasper 10 is shown comprising at the proximal end a handle 20, and at the distal end a grasper 40. Grasper 40 may be useful for multiple types of surgical procedures and grasper 40 may be replaced with any number of other standard surgical instrument end effectors, such as a scissors or forceps to name a few. Specific reference, however, will be made to a grasper for the purpose of suturing or knot tying for explanation purposes only and not for limiting the scope of the claims. Grasper 40 and handle 20 are connected by two, four-bar linkage assemblies and a torsion bar 50 (FIGS. 2 and 3) and an outer tube or shaft 30 enclosing the torsion bar 56.

As shown in FIGS. 2 and 3, torsion bar 50 comprises a central lumen 57 and four slots 52, 53, 54 and 55. Slots 52-55 accommodate four corresponding bars 62, 63, 64 and 65 so that the bars may move freely within the slots. Each bar terminates in a ball or sphere 62a-65a that in turn rotatably couple with a corresponding cavity within a rotational coupler 42 to form a ball and socket type relationship. Coupler 42 forms the proximal end of jaw 40.

Jaw 40 comprises a stationary jaw 46 and a pivotable jaw 44. Pivotable jaw 44 can be held in the open position, away from stationary jaw 14, by a spring (not shown). The four rods 62-65 connect using similar ball and socket joints of connector 28 located at the distal end of handle 20. The combination of rods 62-65, connector 42 and handle connector 28 create two four bar linkages. The first linkage consists of stationary jaw 46, two rods 62 and 64 that are separated by 180 degrees opposite each other on connector 42, and handle connector 28. The second linkage consists of connector 42 and the other two rods 63 and 65 that are opposite each other. A cable (not shown) connects to handle 20 and jaw linkage 48 via lumen 57 to open and close pivotable jaw 44. Jaw linkage 48 is an assembly to pivotally rotate jaw 44 relative to jaw 46 and operatively connects with handle portion 22. Handle portion 22 rotates with respect to handle portion 24 via link 23 to cause the cable to open and close jaw portion 44. Both the jaw linkage 48 and handle linkage 23 are common design features and well known to those skilled in the medical device arts.

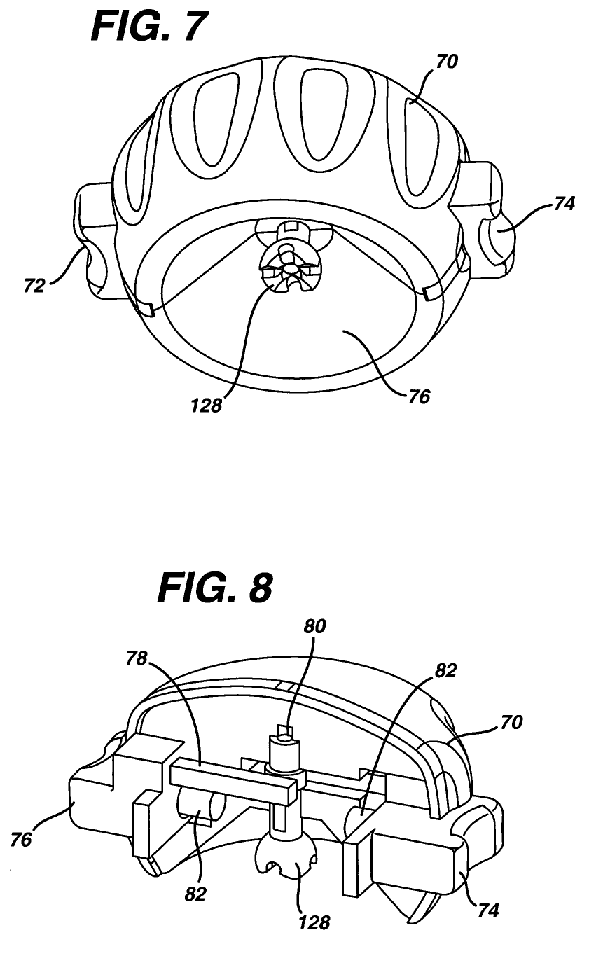

Referring now to FIGS. 6 through 9 there is shown an alternate embodiment to handle 20. In this embodiment a generally spherical handle 70 is configured to fit within the palm of a surgeon's hand. In this embodiment, the surgeon is able to angulate the jaw 40 by grasping handle 70 within the surgeon's palm and articulate his wrist to the desired angular placement of jaw 40. Located on either side of handle 70, 180 degrees apart, are two button/triggers 72 and 74 for actuating pivot jaw 44. Handle 70 defines a cavity 76 comprising a ball and socket coupler 128 that is fixedly attached to handle 70 and a rack 78 and pinion 80 combination. Coupler 128 is a similar ball and socket configuration as couplers 28 and 42 and interface with rods 62-65 and the corresponding ball configuration.

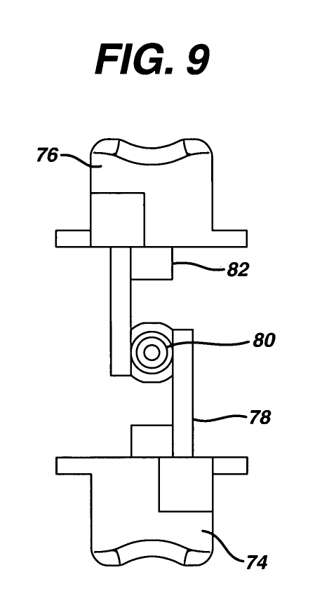

FIGS. 8 and 9 illustrate that by depressing triggers 76 and 74 the linear motion of racks 78 cause pinion 80 to rotate. A cable (not shown), such as a speedometer cable or the like is attached to the pinion 80 and the distal end of the cable is attached to a screw thread, which is operatively attached to pivotable jaw 44. When triggers 76 and/or 74 are depressed, pinion 80 rotates and causes the cable to actuate the screw thread to open jaw 44. When the triggers are released springs 82 force pinion 80 to rotate in the opposite direction and cause the screw thread to close jaw 44.

While the present invention has been illustrated by description of several embodiments, it is not the intention of the applicant to restrict or limit the spirit and scope of the appended claims to such detail. Numerous variations, changes, and substitutions will occur to those skilled in the art without departing from the scope of the invention. Moreover, the structure of each element associated with the present invention can be alternatively described as a means for providing the function performed by the element. Accordingly, it is intended that the invention be limited only by the spirit and scope of the appended claims.

1. A surgical instrument comprising:
a) a handle having a proximal end and a distal end;
b) an end effector having a proximal end and a distal end; and
c) a first and second pair of connector rods connecting the distal end of the handle to the proximal end of the end effector.

2. The surgical instrument of claim 1 further comprising a torsion bar having four channels for operatively receiving the first and second pair of connector rods.

3. The surgical instrument of claim 2, wherein the torsion bar comprises a lumen.

4. The surgical instrument of claim 1, wherein the first and second pair of connector rods are rotatably connected to the distal end of the handle.

5. The surgical instrument of claim 1, wherein the first and second pair of connector rods are rotatably connected to the proximal end of the end effector.

6. The surgical instrument of claim 2 further comprising an elongated outer tube extending from the handle, the outer tube defining a longitudinal axis and having a proximal end and a distal end and a lumen extending therethrough for accepting the torsion bar.

7. The surgical instrument of claim 1, wherein the handle is a generally spherically-shaped member.

8. The surgical instrument of claim 7, wherein the handle comprises a connector element for rotatably receiving the first and second pair of connector rods.

9. The surgical instrument of claim 8, wherein the connector element rotatably connects to at least one operator interface.

10. The surgical instrument of claim 9, wherein the operator interface is a pushbutton.

11. The surgical instrument of claim 9, wherein the connector rotatably connects to the operator interface by a rack and pinion.

12. The surgical instrument of claim 9, wherein the operator interface is spring biased.
